(19) United States
(12) Patent Application Publication
(10) Pub. No.: US 2024/0158409 A1
(43) Pub. Date: May 16, 2024

(54) PROCESS FOR PREPARING A MELAMINE CONDENSATION PRODUCT

(71) Applicant: BASF SE, Ludwigshafen am Rhein (DE)

(72) Inventors: Rene KOENIG, Ludwigshafen am Rhein (DE); Frank NIED, Ludwigshafen am Rhein (DE)

(21) Appl. No.: 18/279,903

(22) PCT Filed: Feb. 24, 2022
(86) PCT No.: PCT/EP2022/054611

§ 371 Date: Sep. 1, 2023

(30) Foreign Application Priority Data
Mar. 4, 2021 (EP) 21160664.5

(51) Int. Cl. C07D 487/16; C09K 21/10

(52) U.S. Cl. CPC C07D 487/16; C09K 21/10

(57) ABSTRACT

A process for preparing a purified product consisting essentially of a melamine condensation product or a salt thereof is provided, the process comprising the steps of a) contacting a raw product comprising a melamine condensation product and an inorganic catalytic residue with an acid having a pKₐ≤3 to form an acidic mixture; b) separating the inorganic catalytic residue from the acidic mixture of step a), and c) obtaining a purified product consisting essentially of a melamine condensation product, wherein the content of the inorganic catalytic residue is at most 2 wt %, based on the total weight of the purified product. The purified product, obtainable by said process, is suitable for use as a flame retardant, especially in plastics, or as a regenerator for nitriding a salt bath.

The present invention relates to a process for preparing a purified product consisting essentially of a melamine condensation product or a salt thereof from a raw product comprising a melamine condensation product and an inorganic catalytic residue using an acid having a pKₐ≤3. Further, the invention relates to a purified product, obtainable by said process, a new polymorph of a compound of formula (I) and to the use of the purified product, obtainable by said process, as a flame retardant, especially in plastics, or as a regenerator for nitriding a salt bath.

### BACKGROUND OF THE INVENTION

Melamine is mainly produced on an industrial scale from urea, either without a catalyst under high pressure or with a catalyst in a low-pressure process. Typical low-pressure processes utilize a fluidized bed at pressures from atmospheric to about 1 MPa and temperatures of 390-410° C. The fluidizing gas is either ammonia or an ammonia-carbon dioxide mixture formed during the course of reaction. Catalysts, for example, include alumina or materials of the silica-alumina type.

The BASF process is a one-stage, low pressure, catalytic vapor-phase process, wherein molten urea is fed to the fluidized catalytic bed reactor at 395-400° C. and atmospheric pressure. A mineral based on aluminum and silicon oxides is used as catalyst, and fluidization is accomplished with an NH₃—CO₂ mixture. The gas leaving the reactor is a mixture of gaseous melamine and condensation products of melamine, for example, melem, and unreacted urea (in the form of its decomposition products isocyanic acid and ammonia) and ammonia and carbon dioxide and additionally entrained catalyst fines.

The gas mixture is cooled to a temperature at which melamine condensation products like melem or higher condensation products as a by-product crystallize. Said precipitated melamine condensation products in the form of a fine powder are removed together with the entrained catalyst fines in adjacent gas filters.

The filtered gas mixture is cooled in a crystallizer to about 190-200° C., wherein more than 98% of the melamine crystallizes as fine crystals.

The composition of the by-product comprises about 50 to 90 wt % of an organic material of carbon, nitrogen and hydrogen and about 50 to 10 wt % of an inorganic material resulting from catalyst fines.

Due to the inorganic material the composition of the by-product is unfavorable and limits a direct application. Coloring and/or undesired mechanical effects may be of disadvantage for some applications, for example, when used as a plastic additive. Therefore, the by-product is usually disposed and incinerated.

Melamine is a molecule which is of particular interest in many applications, for example, in plastics, varnishes, concrete additives or flame retardants. For example, Melapur® is a flame retardant based on melamine.

REG1®, commercially available from Durferrit GmbH, is suitable as a regenerator for nitriding salt baths containing melem in an amount of about half to one third and melon in an amount of about half to two thirds.

Starting from melamine several molecular or polymeric species with interesting properties may be synthesized. By condensation of melamine at elevated temperatures a variety of products may be successively formed, for example: melam, a compound of formula (II); melem, a compound of formula (I) or poly(aminoimino)heptazine, also called melon. B. V. Lotsch et al, Chem. Eur. J. 2007, 13, 4969-4980, describes melon as composed of layers made up from infinite 1D chains of NH-bridged melem monomers, i.e., a condensation product of idealized formula (III).

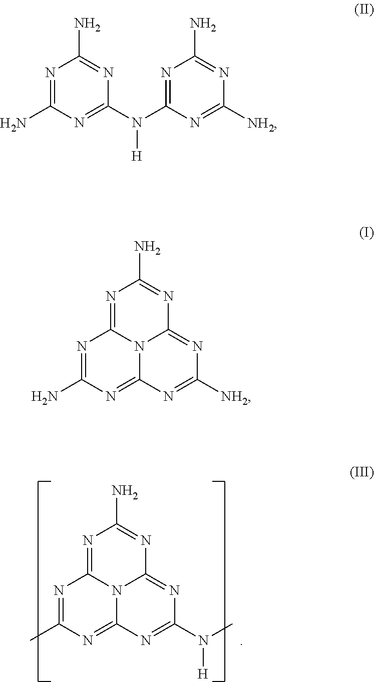

Melem or related melamine condensates like melon may be used as pure substance as flame retardants in plastics. Due to higher thermal stabilities than melamine higher processing temperatures may be achieved which are, for example, needed in processing polyamide or polybutylene terephthalate.

JP 2003-272451 A discloses a flat cable sheating with a flame retardant adhesion layer, wherein inter alia melam sulfate or melem sulfate is mentioned as a component in a flame retardant composition.

JP 2000-198907 A discloses a flame retardant epoxy resin composition, wherein inter alia melam polyphosphate or melem polyphosphate is mentioned as flame retardant.

WO 00/56840 A1 discloses a flame retardant composition comprising, for example, 1 wt % of melamine, 0.1 wt % of melam, 86 wt % of melem and 12.9 wt % of higher condensation products of melamine and/or melem. The composition is prepared by heating melamine as starting material and derived from a gas-phase process at 450° C. within 80 sec in an extruder. The composition is described as a white to pale beige solid having a volatile content of 0.2 wt % and having good burning behavior in various polymeric compositions.

CN 105289721 A discloses a process for preparing melem, wherein urea is sintered at 400-500° C. for 1.5 to 10 hours under argon atmosphere with a specific heating and cooling rate.

SU 707917 discloses a process for preparing melem by pyrolysis of 70% cyanuric acid and 30% of melamine at 380° C. for 1 hour. Additional melem is obtained by pyrolysis of the resulting sublimate containing cyanuric acid, ammelide, ammeline and melamine. The starting material is separated from aqueous waste obtained in melamine production.

Said preparation processes starting from pure substances, e.g., urea or melamine, require, for example, an additional pyrolysis step. The syntheses are often connected with melamine sublimation.

On the other hand, melamine preparation processes are optimized process-wise, in order to avoid side products like melem and higher condensation products and to increase the yield of melamine.

WO 2005/068440 A1 discloses a process for increasing the yield of melamine and improving the dust separation in the melamine production from urea in fluidized-bed catalytic processes, wherein the process gas comprising melamine is transferred into a filter reactor, wherein the nitrogen-containing by-products are reconverted to melamine, and catalyst fines are removed.

A. Sattler et al., Z. Anorg. Allg. Chem. 2005, 631, 2545-2554, discloses syntheses of a melem-adduct C₆N₇(NH₂)₃·H₃PO₄ and of melemium salts (H₂C₆N₇(NH₂)₃)SO₄·H₂O and (HC₆N₇(NH₂)₃ClO₄·H₂O by reaction of melem with the corresponding acids in highly diluted aqueous solution. Melem was prepared by heating melamine at a heating rate of 1° C./min to 460° C. for at least 5 hours under argon atmosphere in a sealed glass ampoule.

B. Juergens et al., J. Am. Chem. Soc. 2003, 125, 10288-10300, discloses a synthesis of melem by heating melamine in a sealed glass ampule to 450° C. to yield a white-beige powder as melem.

Recently, melem and melon gained also interest as directly used or as a precursor for so-called C₃N₄-like materials due to its catalytic and/or semiconducting/electronic properties. H. B. Zheng et al., J. Mater. Chem. C, 2017, 5, 10746-10753 discloses a metal-free blue-emitting material which is described as rod-like structured melem GT, obtained by heating melamine in air at 400° C. forming melem AM, followed by treating said melem with nitric acid and ethylene glycol.

Hence, melem or melon is usually prepared by heating up melamine in inert conditions to 400-600° C., which is not suitable on an industrial scale since melamine decomposes simultaneously and predominantly to ammonia and cyanamide. Thus, a direct access to melamine condensation products is limited.

Accordingly, there is still a need for an easily available synthetic route to a melamine condensation product. The process should be economic and flexible, thus allowing, for example, for the use of an easily available by-product of an industrial process as starting material and avoiding the disposal thereof, and for an easy preparation of melamine condensation products suitable for use as a plastic additive.

Therefore, it is an object of the present invention to provide a process for preparing a melamine condensation product, which is economic and flexible, for example, by using an easily available starting material, in particular, by partially recovering a by-product of a process for preparing melamine.

Further, it is an object of the present invention to provide a melamine condensation product with tailorable properties, concerning, for example, kind of product, purity and/or morphology.

Further, it is an object of the present invention to provide a process for preparing a melamine condensation product which is suitably used as a plastic additive, especially as a flame retardant, or as a regenerator for nitriding a salt bath.

### SUMMARY OF THE INVENTION

It has now been found that a melamine condensation product associated with a catalytic residue may be easily separated from the catalytic residue in order to obtain a purified melamine condensation product or a salt thereof.

Accordingly, in a first aspect the invention relates to a process for preparing a purified product consisting essentially of a melamine condensation product or a salt thereof, the process comprising the steps of
a) contacting a raw product comprising a melamine condensation product and an inorganic catalytic residue with an acid having a pKₐ≤3 to form an acidic mixture;
b) separating the inorganic catalytic residue from the acidic mixture of step a), and
c) obtaining a purified product consisting essentially of a melamine condensation product or salt thereof,
wherein the content of the inorganic catalytic residue of the purified product is at most 2 wt %, based on the total weight of the purified product.

In a further aspect, the invention relates to a melamine condensation product or salt thereof, obtainable by a process, as defined in any aspect herein.

In a further aspect, the invention relates to a compound of formula

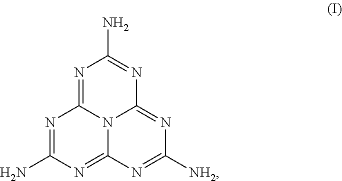 [SMILES: NC1=NC2=NC(N)=NC3=NC(N)=NC(=N1)N23]

wherein the compound is characterized by a X-ray powder diffraction pattern having one or more peaks at a 2Θ angle)(°) of about 10.1±0.2, 10.9±0.2 and 28.0±0.2° using CuK_{α} radiation.

In a further aspect, the invention relates to the use of a purified product consisting essentially of a melamine condensation product or a salt thereof, obtainable by a process, as defined in any aspect herein, or of a compound of formula (I), as defined in any aspect herein, as a flame retardant, preferably in plastics.

In a further aspect, the invention relates to the use of a purified product consisting essentially of a melamine condensation product, obtainable by a process, as defined in any aspect herein, or of a compound of formula (I), as defined in any aspect herein, as a regenerator for nitriding a salt bath.

### DETAILED DESCRIPTION OF THE INVENTION

The term “purified product”, as used herein, means a product consisting essentially of a melamine condensation product or a salt thereof having a content of inorganic material, i.e., an inorganic catalytic residue, of at most 2 wt %, based on the total weight of the purified product.

The term “a salt of a melamine condensation product”, as used herein, includes a salt or an adduct, which is formed from a melamine condensation product and an acid, wherein there is no complete proton transfer from the acid to the melamine condensation product molecule in the adduct, and wherein there is a complete proton transfer to the melamine condensation product molecule in the salt.

The term “a salt of melem”, as used herein, includes a salt or an adduct, which is formed from melem and an acid, wherein there is no complete proton transfer from the acid to the melem molecule in the adduct, and wherein there is a complete proton transfer to the melem molecule in the salt.

The term “a salt of melon”, as used herein, includes a salt or an adduct, which is formed from melon and an acid, wherein there is no complete proton transfer from the acid to melon, the condensation product of formula (III), in the adduct, and wherein there is a complete proton transfer from the acid to melon, the condensation product of formula (III).

The term “consisting essentially of”, as used herein for the purified product, means that the purified product comprises up to 8 wt % of components other than one or more melamine condensation products, preferably 0 to 5 wt %, more preferably 0 to 3 wt %, most preferably 0 to 2 wt %, based on the total weight of the purified product, wherein the content of the inorganic catalytic residue is at most 2 wt %. Said other components may be, for example, ureido melamine, cyanuric acid, ammeline, ammelide or salts or adducts thereof or an adduct of melamine and cyanuric acid.

The term “metal”, as used herein for the component metal oxide, metal oxyhalide or metal halide of the inorganic catalytic residue, also includes silicon.

The term “combination thereof”, as used herein for metal oxides, includes a blend of two or more of metal oxides, a mixed metal oxide of two or more metal oxides and a blend of one or more metal oxides and one or more mixed metal oxides.

The term “combination thereof”, as used herein, means a blend of two, three or more of the respective components.

As used herein, the singular forms of the articles “a”, “an” and “the” include plural forms unless the content clearly dictates otherwise.

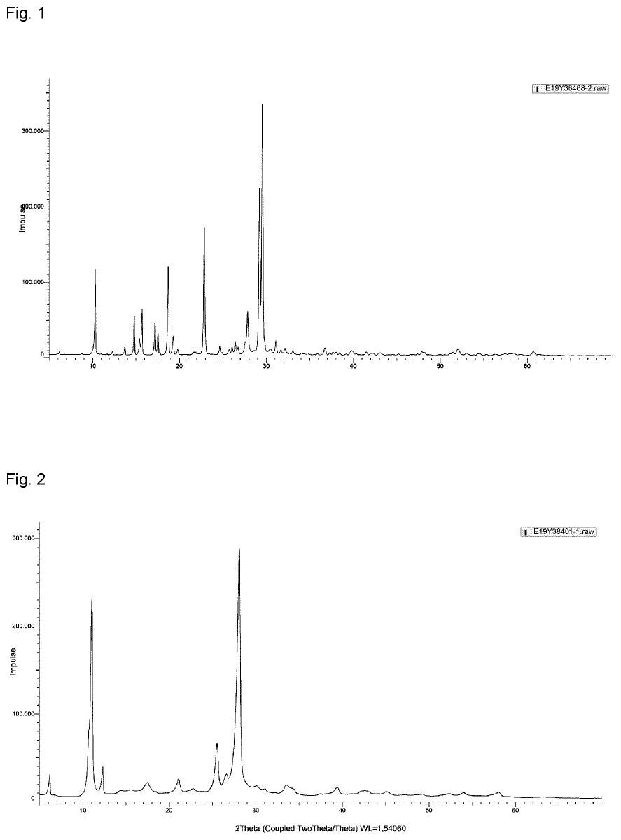

FIG. 1 is a graph showing a powder X-ray diffraction (XRD) spectrum of the product obtained in Example 1.

FIG. 2 is a graph showing a powder X-ray diffraction (XRD) spectrum of the product obtained in Example 2.

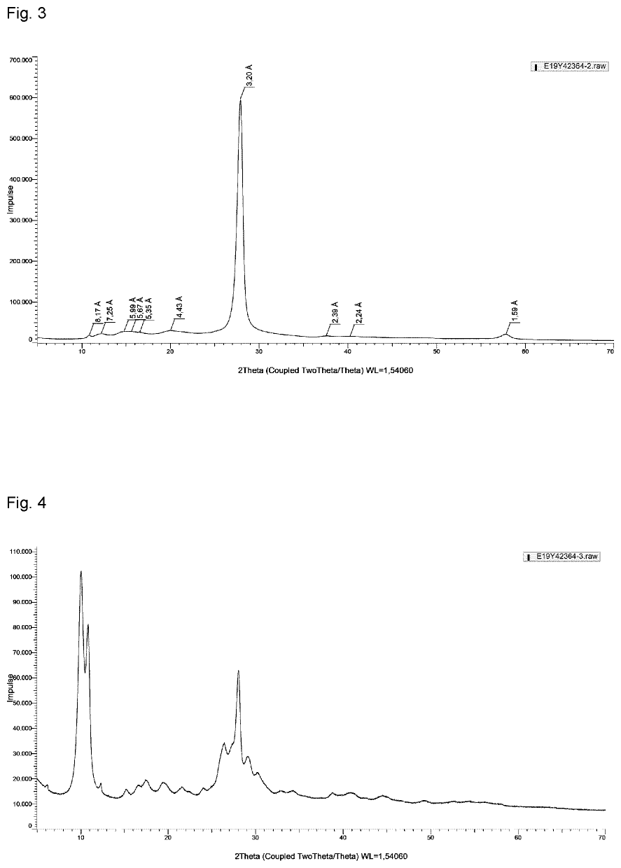

FIG. 3 is a graph showing a powder X-ray diffraction (XRD) spectrum of the product obtained in Example 3.

FIG. 4 is a graph showing a powder X-ray diffraction (XRD) spectrum of the product obtained in Example 4.

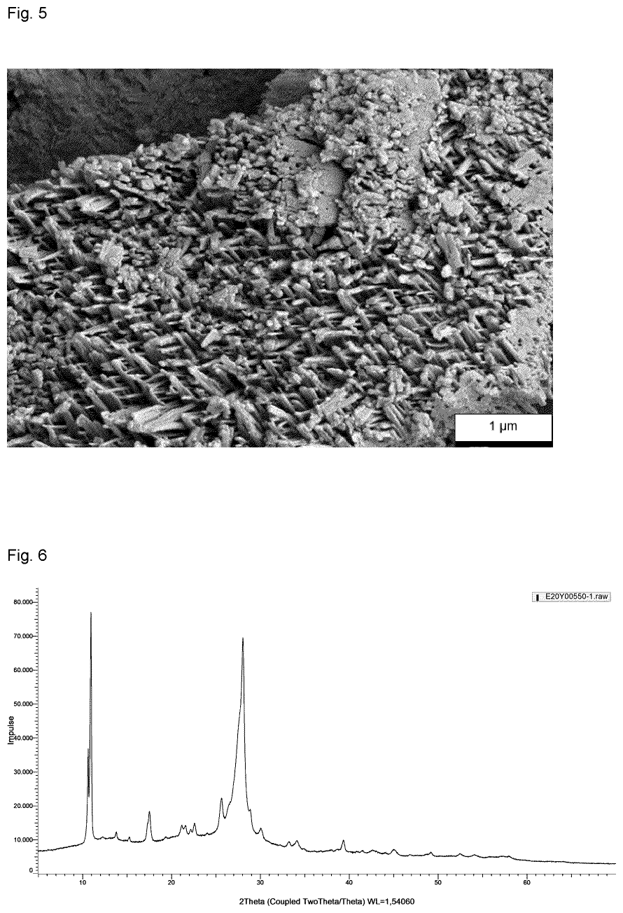

FIG. 5 is a scanning electron microscope (SEM) image showing the product obtained in Example 4.

FIG. 6 is a graph showing a powder X-ray diffraction (XRD) spectrum of the product obtained in Example 5.

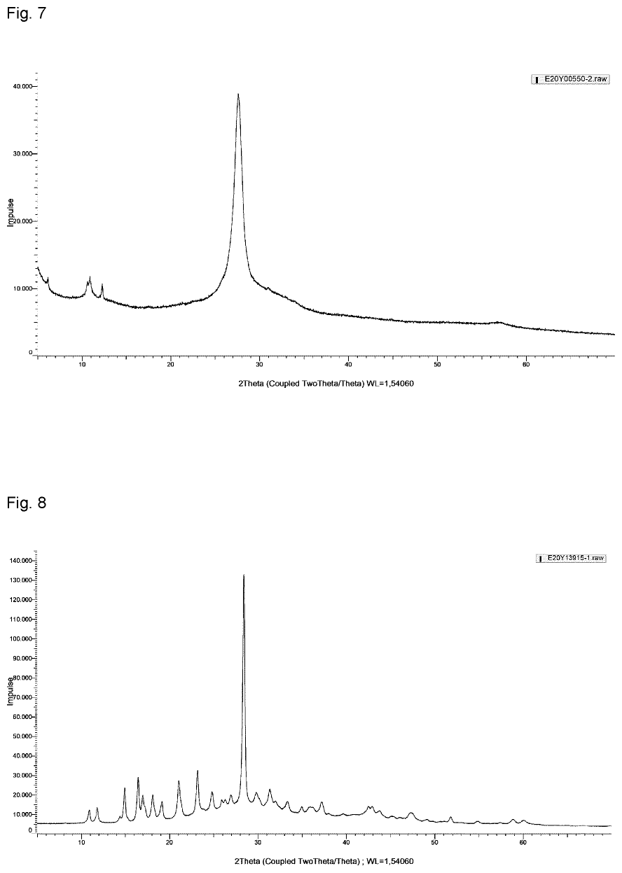

FIG. 7 is a graph showing a powder X-ray diffraction (XRD) spectrum of the product obtained in Example 6.

FIG. 8 is a graph showing a powder X-ray diffraction (XRD) spectrum of the product obtained in Example 7.

The instant process may provide a purified product consisting essentially of a melamine condensation product or a salt thereof having only a minor content of inorganic material derived from a catalytic residue.

The purified product consisting essentially of a melamine condensation product or a salt thereof may comprise one or more melamine condensation products, for example, melem, a salt of melem, melon, a salt of melon, melam, a salt thereof, a further melamine condensation product or salt thereof, which is different from melem, melon or melam or corresponding salts thereof. The purified product may additionally contain water of crystallization.

Melam and further melamine condensation products including salts thereof may only be present in a minor content in the melamine condensation product purified product, usually 0 to 5 wt %, preferably 0 to 3 wt %.

Preferably, the melamine condensation product or a salt thereof comprises melem, a salt thereof, melon, a salt thereof or a combination thereof.

More preferably, the process provides only a specific melamine polycondensation product or salt thereof, i.e., melem, a salt of melem, melon or a salt of melon. Also a combination of a melem and a salt thereof or a combination of melon or a salt thereof may be provided by the instant process.

Thus, more preferably, the melamine condensation product or a salt thereof comprises melem, a salt thereof, melon or a salt thereof. Especially, the melamine condensation product or a salt thereof comprises melem and/or a salt thereof, in particular melem or a salt thereof.

Typically, the raw product comprising a melamine condensation product does not contain a salt or adduct.

Accordingly, in a preferred aspect, the invention relates to a process for preparing a purified product consisting essentially of a melamine condensation product or a salt thereof, wherein the melamine condensation product or salt thereof is selected from melem, a salt thereof, melon, a salt thereof or a combination thereof.

More preferably, the invention relates to a process for preparing a purified product consisting essentially of a melamine condensation product or a salt thereof, wherein the melamine condensation product or salt thereof is selected from melem, a salt thereof, a combination of melem and a salt thereof, melon, a salt thereof or a combination of melon and a salt thereof.

Most preferred, the invention relates to a process for preparing a purified product consisting essentially of a melamine condensation product or a salt thereof, wherein the melamine condensation product or salt thereof is selected from melem, a salt thereof, melon or a salt thereof.

The inorganic catalytic residue may derive from any typical industrial catalysts including, for example, binary metal oxides or consisting of several components and phases. Examples may be complex multicomponent oxides like aluminum silicates, clays, pillared clays or mixed metal oxides.

Typically, the inorganic catalytic residue comprises any metal salt, for example, a salt of a metal selected from the groups I, II, IV, VIII, XIII or XIV of the periodic system.

Preferably, the inorganic catalytic residue may comprise any metal oxide and optionally a metal oxide, which is partially halogenated, and/or a metal halide, for example, an oxide and optionally a partially halogenated oxide and/or a halide of a metal selected from the groups I, II, IV, VIII, XIII or XIV of the periodic system.

Accordingly, in a preferred aspect, the invention relates to a process for preparing a purified product consisting essentially of a melamine condensation product or a salt thereof, wherein the inorganic catalytic residue comprises a metal oxide and optionally a metal oxohalide and/or a metal halide.

Preferably the metal of the oxide, oxohalide or halide is selected from the groups I, II, IV, VIII, XIII or XIV of the periodic system or a combination thereof.

The oxohalide is preferably an oxofluoride or an oxochloride, more preferably an oxofluoride. The halide is preferably a fluoride or a chloride, more preferably a fluoride.

Examples of an oxide of a metal selected from the groups I, II, IV, VIII, XIII or XIV of the periodic system include Na₂O, K₂O, MgO, CaO, Al₂O₃, SiO₂, Fe₂O₃ or TiO₂.

Examples of an oxohalide of a metal selected from the groups I, II, IV, VIII, XIII or XIV of the periodic system include FeOCl, magnesium oxofluorides or oxochlorides or aluminium oxofluorides or oxochlorides.

Examples of a halide of a metal selected from the groups I, II, IV, VIII, XIII or XIV of the periodic system include MgCl₂, AlCl₃, MgF₂ or AlF₃.

Especially preferred is an inorganic catalytic residue comprising a metal oxide and optionally a metal oxofluoride and/or a metal fluoride.

Accordingly, in a preferred aspect, the invention relates to a process for preparing a purified product consisting essentially of a melamine condensation product or a salt thereof, wherein the inorganic catalytic residue comprises a metal oxide selected from Na₂O, K₂O, MgO, CaO, Al₂O₃, SiO₂, TiO₂, Fe₂O₃ or a combination thereof.

The purified product has only a minor content of an inorganic catalytic residue, which is at most 2 wt %, based on the total weight of the purified compound.

Such minor content is not deleterious for the property of flame retardancy of the purified product as well as for the polymeric composition comprising the purified product under processing conditions.

Preferably, the purified product has a content of an inorganic catalytic residue of at most 1.8 wt %, based on the total weight of the purified product, more preferably at most 1.7 wt %.

Accordingly, in a further preferred aspect, the invention relates to a process for preparing a purified product consisting essentially of a melamine condensation product or a salt thereof, wherein the content of the inorganic catalytic residue is at most 1.8 wt %, based on the total weight of the purified product.

Especially, the purified product is essentially free of the inorganic catalytic residue, which means a content of the inorganic catalytic residue of at most 0.2 wt %, based on the total weight of the purified product, in particular, free of an inorganic catalytic residue.

Accordingly, in a more preferred aspect, the invention relates to a process for preparing a purified product consisting essentially of a melamine condensation product or a salt thereof, wherein the purified product is essentially free of the inorganic catalytic residue.

The organic/inorganic raw product comprises a melamine condensation product as the organic part and a catalytic residue as the inorganic part. The organic part and the inorganic part may be present in any possible weight ratios.

Usually, the raw product comprises the melamine condensation product in an amount up to 95 wt %, based on the total weight of the raw product, for example, in an amount of about 30 to 95 wt %.

Preferably, the raw product comprises the melamine condensation product in an amount of from 40 to 90 wt %, based on the total weight of the raw product, more preferably 50 to 90 wt %, most preferably 60 to 80 wt %, especially 70 to 80 wt %.

Usually, the raw product comprises the inorganic catalytic residue in an amount up to 60 wt %, based on the total weight of the raw product, for example, in an amount of about 5 to 60 wt %.

Preferably, the raw product comprises the inorganic catalytic residue in an amount of from 10 to 60 wt %, based on the total weight of the raw product, more preferably 10 to 50 wt %, most preferably 20 to 40 wt %, especially 20 to 30 wt %.

Accordingly, in a preferred aspect, the invention relates to a process for preparing a purified product consisting essentially of a melamine condensation product or a salt thereof, wherein the raw product comprises the melamine condensation product in an amount of 50 to 90 wt %, based on the total weight of the raw product, and the inorganic catalytic residue in an amount of 10 to 50 wt %, based on the total weight of the raw product.

The weight ratio of melamine condensation product to inorganic catalytic residue within the raw product may be of from 40:60 to 90:10, preferably 50:50 to 90:10, more preferably 60:40 to 80:20.

The process is usually carried out by the following steps.

In general, step a) includes the treatment of an organic/inorganic raw product comprising a melamine condensation product as the organic part and a catalytic residue as the inorganic part with an acid having a pKa≤3.

The raw product may be used as a solid or as an aqueous slurry. For example, the raw product may be added as a solid to the acid having a pKa≤3.

The raw product may be used as an aqueous slurry. The raw product may be provided as an aqueous slurry. The acid may then be added to the aqueous slurry of the raw product.

Accordingly, in a preferred aspect the invention relates to a process for preparing a purified product consisting essentially of a melamine condensation product or a salt thereof, wherein the raw product is provided as an aqueous slurry or as a solid.

Further preferred is a process for preparing a purified product consisting essentially of a melamine condensation product or a salt thereof is further preferred, wherein the raw product is provided as an aqueous slurry, and the acid having a pkₐ≤3 is added to the aqueous slurry.

Accordingly, further preferred is a process for preparing a purified product consisting essentially of a melamine condensation product or a salt thereof, the process comprising the steps of
a) contacting a raw product comprising a melamine condensation product and an inorganic catalytic residue with an acid having a pKₐ≤3 to form an acidic mixture;
b) separating the inorganic catalytic residue from the acidic mixture of step a), and
c) obtaining a purified product consisting essentially of a melamine condensation product, wherein the content of the inorganic catalytic residue is at most 2 wt %, based on the total weight of the purified product; and
wherein the raw product is provided as an aqueous slurry or as a solid.

The aqueous slurry may be prepared by providing the raw product comprising the melamine condensation product and the inorganic catalytic residue and adding an aqueous solvent to prepare the aqueous slurry. The aqueous solvent contains water and optionally a water-miscible organic solvent, preferably water.

The water-miscible solvent may be a C₁-C₄-alcohol, C₂-C₈-dialcohol or C₃-C₈-polyalcohol. Examples are methanol, ethanol, 2-propanol, n-propanol, n-butanol, ethylene glycol, propylene glycol, 1,4-butanediol, diethylene glycol, triethylene glycol, glycerol or the like. The amount of the water-miscible solvent in the aqueous slurry may be up to 20 weight parts, based on 100 weight parts of water and water-miscible solvent, preferably up to 10 weight parts.

In general, the aqueous solvent is used in an amount of about 0.5 to about 30 parts by weight, based on 1 part by weight of the raw product, preferably 0.8 to 25 parts by weight.

More preferred is an aqueous slurry, which may be prepared by providing the raw product comprising the melamine condensation product and the inorganic catalytic residue and adding water to prepare the aqueous slurry.

Water used is preferably deionized water.

The acid used in step a) may be an inorganic acid or an organic acid having a pKₐ value of 3 or less, determined in water at 25° C., which are usually the standard conditions.

When a polyprotic acid is used, the first pKₐ is relevant. For example, phosphoric acid has a pKₐ₁ of 2.15.

Preferably, the acid has a pKₐ≤2.5.

Accordingly, a process for preparing a purified product consisting essentially of a melamine condensation product or a salt thereof is preferred, wherein the acid has a pKₐ≤2.5.

The acidic mixture obtained in step a) has usually a pH≤1, preferably a pH≤0.5, more preferably a pH of 0 to 0.5.

Accordingly, a process for preparing a purified product consisting essentially of a melamine condensation product or a salt thereof is preferred, wherein the acidic mixture of step a) has a pH of 0 to 0.5.

The inorganic acid may be selected from the group consisting of sulfuric acid (H₂SO₄), oleum (fuming sulfuric acid), nitric acid (HNO₃), orthophosphoric acid (H₃PO₄), polyphosphoric acid, hydrochloric acid (HCl) and perchloric acid (HClO₄).

The inorganic acid may be used in various concentrations in water. A suitable concentration of an inorganic acid is such which provides an acidic mixture of step a) of a pH≤1, preferably a pH≤0.5, more preferably a pH of 0 to 0.5.

Sulfuric acid may be used in various concentrations, for example, in a concentration of from 50 to 98%, preferably 75 to 98%, more preferably, 85 to 98%, most preferably 90 to 97%. Especially preferred is sulfuric acid (96%).

Nitric acid may be used in various concentrations, for example, in a concentration of from 25 to 80%, preferably 40 to 75%.

Phosphoric acid may be used in various concentrations, for example, in a concentration of from 50 to 95%, preferably 60 to 90%, more preferably 70 to 90%. Especially preferred is phosphoric acid (85%).

The organic acid may be selected from the group consisting of methane sulfonic acid (CH₃SO₃H), ethane sulfonic acid (C₂H₅SO₃H), trifluoroacetic acid (F₃CCO₂H), p-toluene sulfonic acid, sulfamic acid and diphenylphosphinic acid, preferably from the group consisting of methane sulfonic acid (CH₃SO₃H), ethane sulfonic acid (C₂H₅SO₃H), trifluoroacetic acid (F₃CCO₂H) and p-toluene sulfonic acid.

Preferably, the acid is selected from the group consisting of H₂SO₄, oleum, HNO₃ (having a concentration of 25-80%), H₃PO₄, H₃PO₃, polyphosphoric acid, HCl, HClO₄, CH₃SO₃H, C₂H₅SO₃H, p-toluene sulfonic acid, sulfamic acid and diphenylphosphinic acid.

Accordingly, in a preferred aspect the invention relates a process for preparing a purified product consisting essentially of a melamine condensation product or a salt thereof, wherein the acid is selected from the group consisting of H₂SO₄, oleum, HNO₃, H₃PO₄, H₃PO₃, polyphosphoric acid, HCl, HClO₄, CH₃SO₃H, C₂H₅SO₃H, F₃CCO₂H, p-toluene sulfonic acid, sulfamic acid and diphenylphosphinic acid.

More preferably, the acid is selected from the group consisting of H₂SO₄, oleum, HNO₃ (having a concentration of 25-80%), H₃PO₄, H₃PO₃, polyphosphoric acid, HCl, HClO₄, CH₃SO₃H, C₂H₅SO₃H, F₃CCO₂H and p-toluene sulfonic acid.

The acid is generally used in excess, based on 1 weight part of the raw product. For example, the acid may be used in an amount of up to 40 weight parts, based on 1 weight part of the raw product.

Preferably, the weight ratio of raw product to acid is 1:1 to 1:40, preferably 1:2 to 1:35. More preferably, the weight ratio of raw product to acid is 1:10 to 1:40, preferably 1:15 to 1:35, when the acid is HNO₃ (having a concentration of 25-80%), and the weight ratio of raw product to acid is 1:1 to 1:20, preferably 1:2 to 1:15.

Accordingly, in a preferred aspect the invention relates to a process for preparing a purified product consisting essentially of a melamine condensation product or a salt thereof, wherein the weight ratio of raw product to acid is 1:1 to 1:40, preferably 1:2 to 1:35.

When the acidic mixture is formed in step a), the resulting mixture may be self-warming or heated to a temperature, which is sufficient to dissolve the organic part of the raw product. Generally, the acidic mixture is heated to a temperature of from 50° C. to the boiling point of the acidic mixture, preferably from 50 to 180° C., more preferably from 60 to 170° C.

Accordingly, in a preferred aspect the invention relates a process for preparing a purified product consisting essentially of a melamine condensation product or a salt thereof, wherein the acidic mixture of step a) is heated to a temperature of from 50 to 180° C.

Usually, the resulting acidic mixture of step a) is subjected to a solid-liquid separation step. The separation step b) may be carried out by any solid-liquid-separation method known in the art. For example, filtration, centrifugation or decanting may be used.

Preferably, the separation step is carried out by filtration, more preferably at an elevated temperature. The temperature used is generally dependent on the acid used in step a).

The separated liquid, obtained in step b), is usually in form of a solution, which may further be diluted with water or a water-miscible protic solvent, for example, methanol, ethanol, isopropanol, acetonitrile or the like, to yield the purified product as a precipitate.

Alternatively, the purified product may be obtained by cooling the separated liquid, obtained in step b), as precipitate or crystalline product.

Optionally, the separated liquid may be further subjected to a further purification step, for example, according to the level of coloration of the medium a stage of de-coloration by treating with charcoal may be carried out.

The separated liquid may be further treated with a base. The base may be added until a predetermined pH value is adjusted. Dependent on the pH, formation of a precipitate may be observed, i.e., the properties of the melamine condensation product or salt thereof may be adjusted.

Accordingly, in a preferred aspect, the invention relates to a process for preparing a purified product consisting essentially of a melamine condensation product or a salt thereof, wherein the purified product is obtained by treating the separated solution of step b) by adding a base to a pH up to 14.

Generally, any base may be used to adjust a predetermined pH value in order to precipitate the desired purified product. Usually, a water-soluble base is used.

Preferably, the base may be selected from a hydroxide, a hydrogen carbonate, a carbonate or ammonia.

More preferably, the base may be selected from an alkali hydroxide, like NaOH or KOH, alkali hydrogen carbonate, like NaHCO₃ or KHCO₃, alkali carbonate, like Na₂CO₃ or K₂CO₃, or aqueous ammonia (NH₄OH).

The base is usually used as an aqueous solution. The base may be used in various concentrations. For example, ammonia may be used in a concentration of 3-30% in water.

Depending on the acid and the adjusted pH value by a base, various purified products consisting essentially of a melamine condensation product or a salt thereof may be obtained.

Preferably, the salt of the melamine polycondensation product is a phosphate, a sulfate or a nitrate, more preferably a phosphate, sulfate or nitrate of melem.

The instant process may be part of a process for preparing melamine by a catalytic, low pressure process from urea, typically at a temperature of from 380 to 420° C.

The raw product comprising a melamine condensation product or a salt thereof and an inorganic catalytic residue may be obtained as a by-product in a process for preparing melamine. The inorganic catalytic residue may be derived from any catalyst suitable in a process for preparing melamine from urea.

Accordingly, a process for preparing a purified product consisting essentially of a melamine condensation product or a salt thereof is preferred, wherein the raw product comprising a melamine condensation product or a salt thereof and an inorganic catalytic residue is obtainable by a process of preparing melamine, especially from urea at a temperature 380° C., in particular of from 380 to 420° C.

Thus, the invention relates to a process for preparing a purified product consisting essentially of a melamine condensation product or a salt thereof, the process comprising the steps of
a) contacting a raw product comprising a melamine condensation product and an inorganic catalytic residue with an acid having a pKₐ≤3 to form an acidic mixture;
b) separating the inorganic catalytic residue from the acidic mixture of step a), and
c) obtaining a purified product consisting essentially of a melamine condensation product,
wherein the content of the inorganic catalytic residue is at most 2 wt %, based on the total weight of the purified product, and
the raw product is obtainable by a process of preparing melamine from urea at a temperature 380° C., preferably of from 380 to 420° C.

In a further aspect, the invention relates to a melamine condensation product or a salt thereof, obtainable by a process, as defined herein in any aspect.

Accordingly, in a further aspect, the invention relates to a melamine condensation product or a salt thereof, obtainable by a process, the process comprising the steps of
a) contacting a raw product comprising a melamine condensation product and an inorganic catalytic residue with an acid having a pKₐ≤3 to form an acidic mixture;
b) separating the inorganic catalytic residue from the acidic mixture of step a), and
c) obtaining a purified product consisting essentially of a melamine condensation product,
wherein the content of the inorganic catalytic residue is at most 2 wt %, based on the total weight of the purified product.

Preferably, the melamine condensation product or salt thereof is melem, wherein the product is obtainable by treating the separated solution of step b) by adding a base to a pH of 9 to 11.

More preferably, the acid used in step a) is an organic acid, especially selected from the group consisting of methane sulfonic acid (CH₃SO₃H), ethane sulfonic acid (C₂H₅SO₃H), trifluoroacetic acid (F₃CCO₂H) and p-toluene sulfonic acid.

Accordingly, in a preferred aspect, the invention relates to a melamine condensation product or salt thereof, which melamine condensation product or salt thereof is a compound of formula (I) or a salt thereof, obtainable by a process, the process comprising the steps of
a) contacting a raw product comprising a melamine condensation product and an inorganic catalytic residue with an acid having a pKₐ≤3, especially pKₐ≤0.5, to form an acidic mixture;
b) separating the inorganic catalytic residue from the acidic mixture of step a), and
c) treating the separated solution of step b) by adding a base to a pH of 9 to 11.

More preferably, the raw product is provided as an aqueous slurry.

Especially preferred, the acid has a pKₐ≤0,5 and is an organic acid selected from the group consisting of CH₃SO₃H, C₂H₅SO₃H, F₃CCOOH and p-toluene sulfonic acid, most preferably an organic acid of a pKₐ≤0, selected from the group consisting of CH₃SO₃H, C₂H₅SO₃H and p-toluene sulfonic acid, especially selected from the group consisting of CH₃SO₃H and C₂H₅SO₃H.

The instant process may provide a novel polymorph of melem.

Thus, in a further aspect, the invention relates to a compound of formula

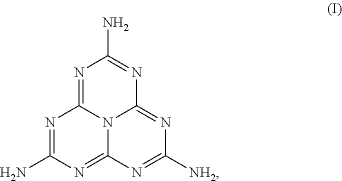 [SMILES: NC1=NC2=NC(N)=NC3=NC(N)=NC(=N1)N23]

wherein the compound is characterized by a X-ray powder diffraction pattern having one or more peaks at a 2Θ angle (°) of about 10.1±0.2, 10.9±0.2 and 28±0.2 using CuK_{α} radiation.

The compound of formula (I) having the above X-ray powder diffraction pattern is a novel polymorph of melem. The pale-yellow product has a rod-shaped morphology, as shown in FIG. 5, of high crystallinity and purity.

Preferred is a compound of formula

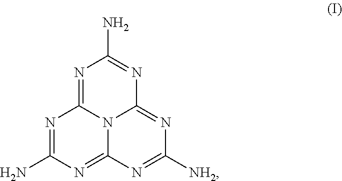 [SMILES: NC1=NC2=NC(N)=NC3=NC(N)=NC(=N1)N23]

wherein the compound is characterized by a X-ray powder diffraction pattern having one or more peaks at a 2Θ angle (°) of about 10.1±0.2, 10.9±0.2 and 28±0.2 using CuK_{α} radiation, and which compound is obtainable by a process, the process comprising the steps of
a) contacting a raw product comprising a melamine condensation product and an inorganic catalytic residue with an acid having a pKₐ≤3, especially pKₐ≤0.5, to form an acidic mixture;
b) separating the inorganic catalytic residue from the acidic mixture of step a), and
c) treating the separated solution of step b) by adding a base to a pH of 9 to 11, wherein the raw product is preferably provided as an aqueous slurry.

More preferred, the acid has a pKₐ≤0,5 and is an organic acid selected from the group consisting of CH₃SO₃H, C₂H₅SO₃H, F₃CCOOH and p-toluene sulfonic acid, most preferably an organic acid of a pKₐ≤0, selected from the group consisting of CH₃SO₃H, C₂H₅SO₃H and p-toluene sulfonic acid, especially selected from the group consisting of CH₃SO₃H and C₂H₅SO₃H.

Due to its low color/chromaticity and the high thermal stability the novel polymorph of formula (I) and the purified compounds are in particular of interest for use as flame retardant in polymer compositions requiring heat-resistant flame retardants, for example, in polyamides, polyimides, polyesters, styrene-containing polymers, epoxy resins, unsaturated polyester resins or polyurethanes.

Further, the novel polymorph and the purified compounds comprising melem or melon are suitable for use as a regenerator for nitriding a salt bath in nitrocarburizing processes of steel, preferably of low alloy steel.

Accordingly, in a further aspect the invention relates to the use of a purified product consisting essentially of a melamine condensation product or a salt thereof, obtainable by the process, as defined in any aspect herein, or the compound of formula (I), wherein the compound is characterized by a X-ray powder diffraction pattern having one or more peaks at a 2Θ angle (°) of about 10.1±0.2, 10.9±0.2 and 28±0.2 using CuK_{α} radiation, as a flame retardant, preferably in plastics.

In a further aspect the invention relates to the use of a compound of formula (I), wherein the compound is characterized by a X-ray powder diffraction pattern having one or more peaks at a 2Θ angle (°) of about 10.1±0.2, 10.9±0.2 and 28±0.2 using CuK_{α} radiation, as a flame retardant, preferably in plastics.

Accordingly, in a further aspect the invention relates to the use of a purified product consisting essentially of a melamine condensation product, obtainable by the process, as defined in any aspect herein, as a regenerator for nitriding a salt bath.

In a further aspect the invention relates to the use of a compound of formula (I), wherein the compound is characterized by a X-ray powder diffraction pattern having one or more peaks at a 2Θ angle (°) of about 10.1±0.2, 10.9±0.2 and 28±0.2 using CuK_{α} radiation, as a regenerator for nitriding a salt bath.

Further, the invention relates to the use of a purified product consisting essentially of a melamine condensation product or a salt thereof, obtainable by the process, as defined in any aspect herein, for preparing a flame retardant polymer composition.

The flame retardant polymer composition may be processed with techniques known to one skilled in the art, for example, injection molding, to form semi-finished products or final products.

The instant process is an easily available synthetic route for preparing a melamine condensation product or a salt thereof, which is suitable for use as a flame retardant or as a regenerator for nitriding a salt bath. The process is economic since the starting material is easily available and may be used to work-up a by-product which is usually be destroyed.

By adjusting the conditions various tailor-made pure melamine condensation products or salts thereof are easily available. Adjusting different pH values and/or using various bases leads to melamine condensation products or salts thereof of different morphologies.

Further, the direct preparation of, for example, a phosphate-containing melamine condensation product enables the material directly to be used as a flame retardant in plastics or as a partial substitute for melamine phosphate.

The amount of residual catalyst of ≤2 wt % is found to not adversely affect the use of a melamine condensation product as a plastic additive, for example, as a flame retardant. Higher amounts would lead to undesired colored plastic products or render plastic compositions brittle.

The melamine condensation products or salts thereof, obtainable by the instant process, are usually thermally stable until at least 500° C. or even higher, which allows high processing temperature when incorporated in plastic compositions, like polyamide or polybutylene terephthalate.

The definitions and preferences given for the pigment mentioned herein-before apply in any combination as well as in any combination for the other aspects of the invention.

The present invention will now be explained in more detail with reference to the following examples. These examples should not be construed as limited. Unless otherwise stated, “%” is always % by weight (wt %).

### EXAMPLES

The raw product comprising a melamine condensation product and an inorganic catalytic residue, which was used as starting material in the Examples, hereinafter referred to as “raw product”, was obtained from a melamine production process and contained approximately 30 wt % of inorganic catalytic residue, which was based on silica-alumina.

The yields can only be determined approximately due to the unknown content of the polycondensation product of melamine in the raw material.

Elemental analysis: C, N and S were measured on analysers from the company Elementar, model Vario MICRO CUBE. Al, Si and P were analyzed after an automated decomposition and ICP OES applying an Spectro BLUE for Al and Si or an Spectro ARCOS for P.

XRD/X-ray powder diffraction pattern: Bruker D8 Advance Bragg-Brentano diffractometer (ambient conditions, Cu Kα; λ=154.06 pm) Range 5°≤2Θ≤70°); the peak having the highest intensity is indicated as “max”.

Thermal behavior: determined by thermogravimetry and differential thermal analysis (TG/DTA): NETSCH STA 449 F3; T range 30-810° C. with heating rate 5° C./min and under inert gas atmosphere

SEM/Scanning electron microscopy: Tescan Vega XM SEM, acceleration voltage of 20 kV using a SE detector

### Example 1

g of the raw product were suspended in 40 g of deionized water under stirring, followed by carefully adding 120 ml of sulfuric acid (96%) in one shot at room temperature (20-25° C.). The resulting self-warming suspension was stirred for 10 min. Then, the hot suspension was filtered off, and the hot filtrate was added to ˜500 ml of deionized water (20-23° C.). During dilution with water a precipitate formed, which was collected by filtration, washed with deionized water and dried at 120° C. to obtain 6.5 g of a colorless/white crystalline product (yield ˜70-80%).

The product was identified as melem-sulfate-hydrate of approximate formula C₆H₆N₁₀·H₂SO₄·¼ H₂O by XRD and elemental analysis.

Elemental analysis: C: 19.1 g/100 g; N: 36.5 g/100 g; S: 7.9 g/100 g (=SO₄˜23.7 g/100 g); Si: 1.1 g/100 g; Al: 0.1 g/100 g.

N/C mass ratio: 1.91 (N/C of melem theor.: 1.94)

XRD: main peaks at 10.3±0.2, 18.7±0.2, 22.9±0.2, 29.1±0.2 and 29.5±0.2 (max)

### Example 2

20 g of the raw product were suspended in 40 g of deionized water under stirring, followed by carefully adding 120 ml of sulfuric acid (96%) in one shot at room temperature. The resulting self-warming suspension was stirred for 10 min. Then, the hot suspension was filtered off, and the hot filtrate was added to ˜200 ml of deionized water and further diluted using an aqueous base (aq. NaOH) until a pH of 7 was adjusted. The resulting precipitate was collected by filtration, washed with deionized water and dried at 120° C. to obtain 6.5 g of a colorless/white crystalline product (yield ˜70-80%).

The product was identified as melem by elemental analysis and XRD.

Elemental analysis: C: 28.1 g/100 g; N: 54.2 g/100 g; Si: 0 g/100 g; Al: 0.02 g/100 g;

N/C mass ratio: 1.93 (N/C of melem theor.: 1.94)

XRD: main peaks at 11.0±0.2 and 28.2±0.2 (max)

TG/DTA: thermally stable up to a temperature of about 510° C., completely degraded at about 690° C.

### Example 3

20 g of the raw product were suspended in 40 g of deionized water under stirring, followed by carefully adding 120 ml of sulfuric acid (96%) in one shot at room temperature. The resulting self-warming suspension was stirred for 10 min. Then, the hot suspension was filtered off, and the hot filtrate was added to ˜500 ml of deionized water and further diluted with sodium hydroxide (25 wt % in water) until a pH of 2 was adjusted. The resulting precipitate was collected by filtration, washed with deionized water and dried at 120° C. to obtain 8 g of a colorless/white solid (yield of 80-90%). The product was identified as melon by XRD and elemental analysis.

Elemental analysis: C: 15.6 g/100 g; N: 27.5 g/100 g; Al: 0 g/100 g; Si: 0 g/100 g; N/C mass ratio: 1.75 (theor.: 1.75 for —(C₆H₃N₉)—)

XRD: main peak at 28.0±0.2

TG/DTA: Thermally stable up to a temperature of about 528° C., completely degraded at about 690° C.

### Example 4

1.5 g of the raw product were suspended in 30 g of deionized water under stirring, followed by adding 20 ml of methane-sulfonic acid in one shot at room temperature. The suspension was heated to the boiling temperature for 10 min. The hot suspension was filtered off, followed by adjusting the pH of the filtrate to 10 by adding ammonia water (conc. ˜5%). The resulting precipitate was collected by filtration, washed with deionized water and dried at 120° C. to obtain 0.8 g of a pale-yellow crystalline product with rod-shaped morphology (yield ˜80%).

The product was identified as melem by XRD and elemental analysis.

Elemental analysis: C: 28 g/100 g; N: 53 g/100 g; Al: 0 g/100 g; Si: 0 g/100 g; N/C mass ratio: 1.89 (N/C melem theor.: 1.94)

XRD: main peaks at 10.1±0.2 (max), 10.9±0.2 and 28±0.2

TG/DTA: thermally stable up to a temperature of about 535° C., completely degraded at about 671-698° C.

### Example 5

10 g of the raw product were suspended in 10 g of deionized water under stirring, followed by adding 30 ml of phosphoric acid (85%) in one shot at room temperature. The resulting suspension was stirred for 30 min at 90° C. Then, the hot suspension was filtered off. Residual colored contaminants were removed from the filtrate with charcoal followed by filtration. The pH of the filtrate was adjusted to 4 by adding an aqueous solution of sodium hydrogen carbonate. The resulting precipitate was collected by filtration, washed with deionized water and dried at 120° C. to obtain a white/colorless weak crystalline product.

The product was identified as a salt of melem comprising phosphate (13 wt %), as found by elemental analysis and XRD.

Elemental analysis: C: 25 g/100 g; N: 48 g/100 g; P: 4.1 g/100 g (=PO₄˜13 g/100 g); Si: 0.03 g/100 g; Al: 0.03 g/100 g;

N/C mass ratio 1.92 (N/C of melem theor.: 1.94)

XRD: main peaks at 10.7±0.2, 11.0±0.2 (max), 27.3±0.2 and 28.1±0.2

### Example 6

The procedure of Example 5 was repeated with the exception that the pH was adjusted to 7. A colorless/white solid was obtained as product.

The product was identified as a salt of melem having 17 wt % of phosphate, as found by elemental analysis and XRD.

Elemental analysis: C: 19.4 g/100 g; N: 37.1 g/100 g; P: 5.4 g/100 g (=PO₄˜17 g/100 g); Si: 0.03 g/100 g; Al: 1.6 g/100 g;

N/C mass ratio 1.91 (N/C of melem theor.: 1.94)

XRD: main peak at 27.8±0.2

### Example 7

5 g of the raw product were suspended in 150 ml of HNO₃ (˜50%). The suspension was stirred for 90 min at 90° C., followed by filtration the hot suspension. The filtrate was added to ˜500 ml of deionized water, the resulting precipitate was collected by filtration, washed with deionized water and dried to obtain a white/colorless crystalline product, which is kept moist.

The product was identified as a salt of melem comprising nitrate by XRD and elemental analysis.

Elemental analysis: C: 13 g/100 g; N: 27.8 g/100 g; Si: 0.02 g/100 g; Al: 0.01 g/100 g;

N/C mass ratio ˜2.14

XRD: main peak at 28.4±0.2

1.-15. (canceled)

16. A process for preparing a purified product consisting essentially of a melamine condensation product or a salt thereof, the process comprising steps of
a) contacting a raw product comprising a melamine condensation product and an inorganic catalytic residue with an acid having a pKₐ≤3 to form an acidic mixture;
b) separating the inorganic catalytic residue from the acidic mixture of step a), and
c) obtaining a purified product consisting essentially of a melamine condensation product, wherein the content of the inorganic catalytic residue is at most 2 wt %, based on the total weight of the purified product.

17. The process according to claim 16, wherein the melamine condensation product or salt thereof is selected from melem, a salt thereof, melon, a salt thereof or a combination thereof.

18. The process according to claim 16, wherein the inorganic catalytic residue comprises a metal oxide, and optionally a metal oxohalide and/or a metal halide.

19. The process according to claim 16, wherein the inorganic catalytic residue comprises a metal oxide selected from Na₂O, K₂O, MgO, CaO, Al₂O₃, SiO₂, TiO₂, Fe₂O₃ or a combination thereof.

20. The process according to claim 16, wherein the raw product comprises the melamine condensation product in an amount of 50 to 90 wt %, based on the total weight of the raw product, and the inorganic catalytic residue in an amount of 10 to 50 wt %, based on the total weight of the raw product.

21. The process according to claim 16, wherein the acid has a pKₐ≤2.5.

22. The process according to claim 16, wherein the acid is selected from the group consisting of H₂SO₄, oleum, HNO₃, H₃PO₄, H₃PO₃, polyphosphoric acid, HCl, HClO₄, CH₃SO₃H, C₂H₅SO₃H, F₃CCO₂H, p-toluene sulfonic acid, sulfamic acid and diphenylphosphinic acid.

23. The process according to claim 16, wherein the weight ratio of raw product to acid is 1:1 to 1:40.

24. The process according to claim 16, wherein the purified product is obtained by treating the separated solution of step b) by adding a base to a pH up to 14.

25. The process according to claim 16, wherein the raw product comprising a melamine condensation product or a salt thereof and an inorganic catalytic residue is obtainable by a process of preparing melamine.

26. A melamine condensation product or a salt thereof, obtained by the process as defined in claim 16.

27. The melamine condensation product or salt thereof according to claim 26, which product is melem, wherein the product is obtained by treating the separated solution of step b) by adding a base to a pH of 9 to 11.

28. A compound of formula 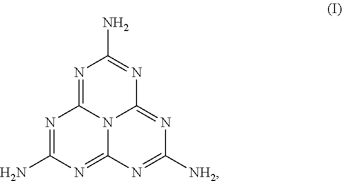 [SMILES: NC1=NC2=NC(N)=NC3=NC(N)=NC(=N1)N23]
wherein the compound is characterized by a X-ray powder diffraction pattern having peaks at a 2Θ angle (°) of 10.1±0.2, 10.9±0.2 and 28±0.2 using CuK_{α} radiation.

29. A method comprising utilizing a purified product consisting essentially of a melamine condensation product or a salt thereof, obtained by the process as defined in claim 16 as a flame retardant.

30. A method comprising utilizing a purified product consisting essentially of a melamine condensation product or a salt thereof, obtained by the process as defined in claim 16 as a regenerator for nitriding a salt bath.
